(19) United States
(12) Patent Application Publication
(10) Pub. No.: US 2026/0167591 A1
(43) Pub. Date: Jun. 18, 2026

(54) DECHLORINATING AND SEPARATING A MIXTURE OF ALKYLATE AND N-BUTANE

(71) Applicant: CHEVRON U.S.A. INC., San Ramon, CA (US)

(72) Inventors: Christine Marie LONG, El Cerrito, CA (US); Huping LUO, Moraga, CA (US); Howard Steven LACHEEN, Richmond, CA (US); Bong Kyu CHANG, Novato, CA (US); Mark D. RASMUSSEN, Farmington, UT (US); Hye-Kyung Cho TIMKEN, Albany, CA (US); Trung PHAM, Fox River Grove, IL (US); Gregory J. SCHRAD, Winfield, IL (US)

(21) Appl. No.: 19/423,733

(22) Filed: Dec. 17, 2025

Related U.S. Application Data
(60) Provisional application No. 63/735,790, filed on Dec. 18, 2024.

(51) Int. Cl. C07C 7/04; C07C 1/30

(52) U.S. Cl. CPC C07C 7/04; C07C 1/30

(57) ABSTRACT

A process is provided for dechlorinating and separating a mixture of n-butane and alkylate. The process includes separating, in a separation zone, a feed stream comprising light hydrocarbons, propane, isobutane, organic chlorides, n-butane, and alkylate with C5+ hydrocarbons into an overheads stream comprising light hydrocarbons, propane, and isobutane and a bottoms stream comprising organic chlorides, n-butane and alkylate; passing the bottoms stream to a catalytic dechlorination zone configured to convert the organic chlorides to HCl and paraffins under catalytic dechlorination conditions, the catalytic dechlorination zone comprising a vessel and being configured to provide a HCl-rich stream, an n-butane-rich stream, optionally an isopentane-rich stream, and an alkylate-rich stream; removing the HCl-rich stream from the catalytic dechlorination zone as a gaseous overheads product; recovering the n-butane-rich stream from the catalytic dechlorination zone as a liquid side-draw product; optionally recovering the isopentane-rich stream from the catalytic dechlorination zone as a side-draw product; and recovering the alkylate-rich stream from the catalytic dechlorination zone as a liquid bottoms product.

### RELATED APPLICATIONS

This application claims the benefit of U.S. Provisional Application No. 63/735,790, filed on Dec. 18, 2024, the disclosure of which is incorporated herein by reference in its entirety.

### TECHNICAL FIELD

The following disclosure relates generally to processes for dechlorinating and separating mixtures of alkylate and normal butane (n-butane).

### BACKGROUND

Alkylation is the reaction between feed hydrocarbons and an alkylating agent. Hydrocarbon alkylation is widely used in the petroleum refining and petrochemical industries to produce a variety of useful acyclic and cyclic hydrocarbon products used as motor fuel, plastic and detergent precursors and petrochemical feedstocks. For example, large amounts of high-octane gasoline are produced commercially by alkylation of isobutane with butenes or propylene. This significantly increases the value of the C4 feed hydrocarbons.

Historically, such alkylation processes have commonly employed hydrofluoric acid (HF) or sulfuric acid (H₂SO₄) as a catalyst. Environmental and safety concerns, among other factors, have led to the development of newer processes utilizing other catalysts. For example, acidic ionic liquids have been utilized as catalysts in hydrocarbon conversion processes, such as alkylation, isomerization, disproportionation, reverse disproportionation, and oligomerization. Ionic liquid catalysts are more environmentally friendly and therefore more desirable than sulfuric acid and hydrofluoric acid.

The use of ionic liquids for alkylation requires the use of hydrogen chloride (HCl) or an HCl precursor as a co-activator/co-catalyst. The HCl can be introduced into the process in several ways, such as the direct vapor phase injection of HCl to the ionic liquid using anhydrous HCl, or the introduction of a liquid organic chloride. Although the vapor phase method is effective, stringent governmental regulations covering the use of HCl gas may make it impractical. The liquid phase method is less effective because organic chlorides do not readily breakdown to HCl and paraffins in the absence of a catalyst.

The primary alkylation products are n-butane and alkylate. The n-butane product stream is typically drawn as a side-draw from a fractionation column, such as an isostripper, deisobutanizer, or depropanizer column. The n-butane can be used as regenerant or as feed or sold as final product. The alkylate product stream is typically drawn separately as a bottoms product from the fractionation column.

Products from the alkylation reactor may contain excess inorganic chloride (HCl) and organic chloride (e.g., butyl chloride) byproducts formed by the reaction of HCl with olefins in the feed, which must be removed from the products to meet specifications and avoid downstream corrosion.

A number of processes have been developed to handle the HCl and organic chlorides in alkylation processes. For example, chloride treaters may be located in product streams to remove the chloride contaminants. Such treaters use adsorbents and may be used to remove chloride from both gas and liquid streams. However, the adsorbents are expensive and labor intensive to change out.

Some recent efforts have focused on processing/treating only the n-butane product stream to remove chlorides. However, treating only the n-butane requires recycle streams between the n-butane column and the isostripper, which requires expensive modifications to the isostripper. Other efforts have focused on processing/treating the isostripper feed which is a significantly larger stream requiring large equipment and thereby incurring greater capital expenditure. In addition to processing larger streams, the isostripper is not a desirable location as it is at a low temperature that would require heating and pressurization.

Accordingly, it would be desirable to have more effective and efficient ways to control both the organic and inorganic chloride levels in the alkylate and n-butane product streams, particularly without requiring recycle streams or modifications to existing equipment.

### SUMMARY

In a first aspect, the present disclosure relates to a process for processing a product stream comprising organic chlorides, n-butane and alkylate, which comprises:
separating, in a separation zone, a feed stream comprising light hydrocarbons, propane, isobutane, organic chlorides, n-butane, and alkylate with C5+ hydrocarbons into an overheads stream comprising light hydrocarbons, propane and isobutane and a bottoms stream comprising organic chlorides, n-butane and alkylate; and passing the bottoms stream to a catalytic dechlorination zone configured to convert the organic chlorides to HCl and paraffins under catalytic dechlorination conditions, the catalytic dechlorination zone comprising a vessel and being configured to provide a HCl-rich stream, an n-butane-rich stream, an alkylate-rich stream; removing the HCl-rich stream from the catalytic dechlorination zone as a gaseous overheads product; recovering the n-butane-rich stream from the catalytic dechlorination zone as a side-draw product; and recovering the alkylate-rich stream from the catalytic dechlorination zone as a liquid bottoms product.

In a second aspect, the present disclosure relates to a process for processing a product stream comprising organic chlorides, n-butane and alkylate, which comprises: separating, in a separation zone, a feed stream comprising light hydrocarbons, propane, isobutane, organic chlorides, n-butane, and alkylate with C5+ hydrocarbons into an overheads stream comprising light hydrocarbons, propane, and isobutane and a bottoms stream comprising organic chlorides, n-butane and alkylate; and passing the bottoms stream to a catalytic dechlorination zone configured to convert the organic chlorides to HCl and paraffins under catalytic dechlorination conditions, the catalytic dechlorination zone comprising a vessel and being configured to provide a HCl-rich stream, an n-butane-rich stream, an isopentane-rich stream, and an alkylate-rich stream; removing the HCl-rich stream from the catalytic dechlorination zone as a gaseous overheads product; recovering the n-butane-rich stream from the catalytic dechlorination zone as a first side-draw product; recovering the isopentane-rich stream as a second side-draw product; and recovering the alkylate-rich stream from the catalytic dechlorination zone as a liquid bottoms product. The recovered n-butane-rich stream may be passed to a butane isomerization unit for conversion to isobutane. The recovered isopentane-rich stream may be recycled back to an alkylation reactor to produce additional alkylate. The recovered alkylate-rich stream may provide an alkylate having a low Reid vapor pressure for use in gasoline blending.

### BRIEF DESCRIPTION OF THE DRAWINGS

FIG. 1 is a schematic process flow diagram according to one or more aspects of the present disclosure.

FIG. 2 is another schematic process flow diagram according to one or more aspects of the present disclosure.

FIG. 3 is yet another schematic process flow diagram according to one or more aspects of the present disclosure.

### DETAILED DESCRIPTION

The term “alkylate” typically refers to branched-chain paraffin. The branched-chain paraffin typically is derived from the reaction of isoparaffin with olefin. More specifically, the term “alkylate” refers to hydrocarbon compositions used for fuel applications having at least 55 normalized vol. % C 5-C10 isoparaffins measured according to ASTM D6730 or having at least 55 vol. % C 5-C10 isoparaffins measured pursuant to ASTM D6733.

As used herein, the term “light hydrocarbons” refers to hydrocarbons having carbon numbers in a range from 1 to 4.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Unless indicated otherwise, overhead lines and bottoms lines refer to the net lines from the column downstream of any reflux or reboil take-off to the column. Stripper columns may omit a reboiler at a bottom of the column and instead provide heating requirements and separation impetus from a fluidized inert media such as steam. The column may be a dividing-wall column. Dividing-Wall Columns (DWC) are distillation columns containing vertical dividing walls that partition a feed zone from one or more side product draw-off zones. The dividing walls can be used to separate the volume used for receiving input feed(s) to the column from the locations for withdrawal of product streams, thereby reducing product contamination. Additionally or alternately, the dividing walls can be used to create separate compartments for withdrawal of multiple product streams with high purity (e.g., greater than 99 wt. %).

As used herein, the term “reactive distillation column” is used conventionally to refer to a distillation column in which both reaction and separation is performed.

As used herein, the term “overheads” can mean a stream withdrawn at or near a top of a column, typically a distillation column.

As used herein, the term “bottoms” can mean a stream withdrawn at or near a bottom of a column, typically a distillation column.

As used herein, the term “side draw” or “side product” can mean a stream withdrawn from an intermediate location within the column, typically a distillation column.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “a component-rich stream” means that the rich stream coming out of a vessel has a greater concentration of the component than the feed to the vessel.

As used herein, the term “rich” means greater than 50%, suitably greater than 75% and preferably greater than 90%

As depicted, process flow lines in the FIGURES can be referred to interchangeably as, for example, lines, pipes, feeds, effluents, products, or streams.

With these general principles in mind, one or more embodiments of the present disclosure will be described with the understanding that the following description is not intended to be limiting.

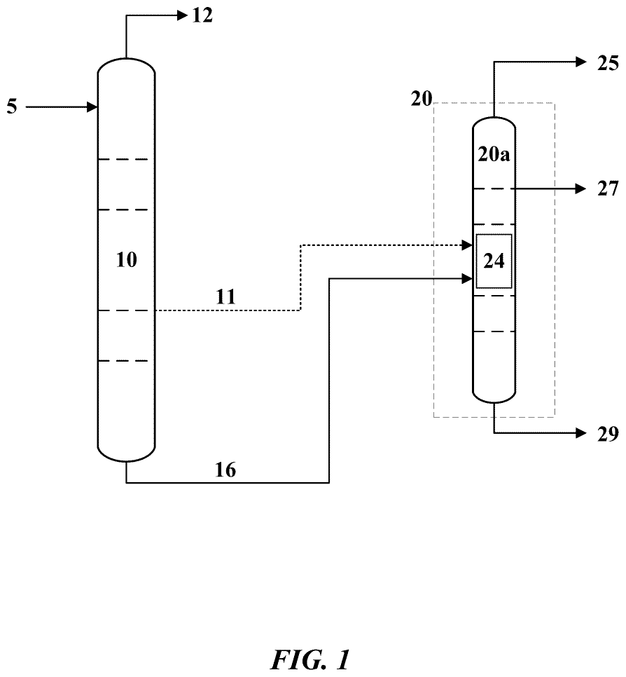

As shown in FIGS. 1, 2 and 3, a feed stream 5 comprising light hydrocarbons, propane, isobutane, n-butane, chlorides, and alkylate with C5+ hydrocarbons is passed to a separation zone 10. The separation zone 10 comprises a fractionation column which may be a deisobutanizer. As is known, in the fractionation column the components of the feed stream will separate. Accordingly, the separation zone 10 is configured to provide a gaseous overheads stream 12 comprising light hydrocarbons, propane and isobutane and a small amount of HCl, and a liquid bottoms stream 16 comprising organic chlorides, n-butane, and alkylate. Separation zone 10 may be configured to operate providing the bottoms stream 16 with sufficient temperature going to the dechlorination zone 20 without the need to add any additional heater or heat exchanger (e.g., by operating separation zone 10 at a slightly higher pressure to have a bottom temperature greater than 275° F.). Optionally, a side-draw stream 11 comprising at least a portion of the n-butane can be recovered from separation zone 10, as typically practiced, and passed separately to catalytic dechlorination zone 20. The gaseous overheads stream comprising light hydrocarbons 12 may be passed to a depropanizer column and processed further as is known in the art.

The feed stream 5 is preferably an effluent from an ionic liquid-catalyzed isoparaffin-olefin alkylation reaction zone. Such reaction zones are known in the art. The paraffin used in the alkylation process preferably comprises an isoparaffin having from 4 to 10 carbon atoms, or 4 to 8 carbon atoms, or 4 to 5 carbon atoms. The olefin used in the alkylation process preferably has from 2 to 5 carbon atoms, or 3 to 5 carbon atoms.

Feed stream 5 comprises an organic chloride contaminant. Representative organic chlorides include, but are not limited to, perchlorides, such as perchloroethylene and carbon tetrachloride and chlorides, such as methyl chloride, methylene chloride, ethyl chloride, dichloroethane, dichloroethylene, trichloroethylene, dichloropropane, dichloropropene, sec-butyl chloride, tert-butyl chloride, and C5+ chlorides. In one aspect, feed stream 5 may have a chloride concentration in a range of 200 wppm to 5000 wppm, alternatively 250 wppm to 4000 wppm, or alternatively 300 wppm to 3000 wppm, as measured by ASTM D4929.

As shown in FIGS. 1, 2 and 3, bottoms stream 16 comprising organic chlorides, n-butane and alkylate is passed to catalytic dechlorination zone 20 that includes one or more vessels. Optionally, a side-draw stream 11 comprising at least a portion of the n-butane can be recovered from separation zone 10 and can be combined with bottoms stream 16 comprising alkylate forming a combined stream which is passed to the catalytic dechlorination zone 20. Alternatively, side-draw stream 11 comprising at least a portion of the n-butane can be recovered from separation zone 10 and passed separately to catalytic dechlorination zone 20. According to the embodiment shown in FIG. 1, the catalytic dechlorination zone 20 includes one vessel 20a that is a reactive distillation column. As is known, the reactive distillation column 20a includes a reactive section 24 containing catalyst that, under appropriate operation conditions, promotes a chemical reaction—in this case the conversion of an organic chloride to gaseous HCl and the corresponding paraffin (e.g., convert tert-butyl chloride to HCl and isobutane). In addition to the conversion of organic chlorides to HCl and the corresponding paraffins, the reactive distillation column 20a also separates the components within the reactive distillation column 20a into at least three component streams, namely, an HCl-rich gaseous overhead stream 25, an n-butane-rich side-draw product stream 27, and a liquid alkylate-rich bottoms stream 29. Optionally, an isopentane-rich side-draw product stream may also be recovered from the reactive distillation column. In some aspects, the reactive distillation column is a dividing-wall reactive distillation column. Optionally, a small treater (caustic or adsorbent) may be added to one or more product streams out of column 20a to remove trace amounts of HCl/organic chloride in case of small chloride slip at trace concentrations.

In the present process, a number of different catalysts can be used for dechlorination. In some aspects, the catalyst may be a solid catalyst selected from silica, alumina, chlorinated alumina, silica-alumina, magnesium oxide, titanium oxide, zinc oxide, zirconium oxide, activated carbon, a zeolite, and any combinations thereof. In one aspect, the catalyst may comprise, consist essentially of, or consist of alumina. In another aspect, the catalyst may comprise, consist essentially of, or consist of a zeolite (e.g., zeolite X). In some aspects, the solid catalyst may also contain a precious metal such as Pt, Pd, Ru, Re, or any combinations thereof. The solid catalyst may be in the form of a mesh, pellet, or sphere.

In certain other aspects, the catalyst comprises an ionic liquid. Ionic liquids are salts comprised of organic cations and anions which typically melt below about 100° C.

In some aspects, the ionic liquid comprises an organic cation and a halometallate anion and wherein the organic cation comprises an ammonium cation, a pyrrolidinium cation, a pyridinium cation, an imidazolium cation, a phosphonium cation, or a combination thereof.

In some aspects, the ionic liquid comprises an organic cation and a halometallate anion and wherein the halometallate anion comprises a metal selected from Al, Ga, In, Mn, Fe, Co, Ni, Cu, Zn, or a combination thereof, and a halide selected from F, Cl, Br, I, or a combination thereof.

Examples of ammonium cations include, but are not limited to, tetraalkylammonium cations (e.g., tri-C1-C6 alkyl-C2-C10 alkyl ammonium cations, such as trimethyl-n-propylammonium, n-butyl-trimethylammonium, n-hexyl-trimethylammonium, triethyl-methylammonium, tetraethylammonium, n-butyl-triethylammonium, and tetra-n-butylammonium).

Examples of pyrrolidinium cations include, but are not limited to, N, N-dialkylpyrrolidinium cations (e.g., N-C1-C3 alkyl-N-C2-C6 alkyl-pyrrolidinium cations, such as N-methyl-N-propylpyrrolidinium and N-butyl-N-methylpyrrolidinium).

Examples of imidazolium cations include, but are not limited to, 1,3-dialkylimidazolium cations (e.g., 1-C2-C10 alkyl- 3-C1-C3 alkyl-imidazolium cations, such as 1-ethyl-3-methylimidazolium, 1-n-butyl-3-methylimidazolium, 1-n-hexyl-3-methylimidazolium, and 1-n-octyl-3-methylimidazolium).

Examples of pyridinium cations include, but are not limited to, N-alkylpyridinium cations (e.g., N-C2-C6 alkyl-pyridinium cations, such as N-ethylpyridinium and N-butylpyridinium).

Examples of phosphonium cations include, but are not limited to, tetraalkylphosphonium cations (e.g., tri C1-C10 alkyl-C2-C20 alkylphosphonium cations, such as triethyl-pentylphosphonium, tetrabutylphosphonium, and trihexyl-tetradecylphosphonium).

The anion of the ionic liquid comprises a halometallate. Halometallate anions may contain a metal selected from Al, Ga, In, Mn, Fe, Co, Ni, Cu, Zn, or combinations thereof, and a halide selected from F, Cl, Br, I, or combinations thereof. In some aspects, the anion of the ionic liquid comprises a haloaluminate. In some aspects, the anion of the ionic liquid comprises a chloroaluminate. The anion may be formally an anion, or it may be an anion associated with a metal halide. For instance, the anion may be AlCl₄⁻ or Al₂Cl₇⁻ associated with AlCl₃. The anion may be GaCl₄⁻ or Ga₂Cl₇⁻ or Ga₃Cl₁₀⁻ associated with GaCl₃.

In one aspect, the ionic liquid catalyst comprises, consists essentially of, or consists of 1-ethyl-3-methylimidazolium heptachloroaluminate [emim⁺][Al₂Cl₇⁻]. In another aspect, the ionic liquid catalyst comprises, consists essentially of, or consists of N-butylpyridinium heptachloroaluminate [NBuPy⁺][Al₂Cl₇⁻].

The ionic liquid catalyst may be supported or may be fed to the reactive distillation column. Suitable supports include silica, alumina, silica-alumina, magnesium oxide, titanium oxide, zinc oxide, zirconium oxide, activated carbon, a zeolite, and any combinations thereof. When ionic liquid catalyst is fed together with the bottoms stream to the reactive distillation column, the amount of ionic liquid can be adjusted to an effective amount (e.g., 0.001 to 0.1 wt. %, based on a combined weight of the ionic liquid catalyst and bottoms stream).

The conditions for operating the catalytic dechlorination zone can include a temperature of 150° F. to 650° F. (65° C. to 343° C.), such as 200° F. to 600° F. (93° C. to 316° C.), or 250° F. to 550° F. (121° C. to 288° C.). The catalytic dechlorination conditions may include a pressure of 10 to 1000 psig (0.069 to 6.9 MPa gauge), such as 30 to 600 psig (0.206 to 4.13 MPa gauge). A liquid hourly space velocity (LHSV) to the catalytic dechlorination zone may be in the range of 0.1 to 50 h⁻¹, such as 0.5 to 20 h⁻¹. Catalytic dechlorination can be conducted in the presence of hydrogen or in the absence of hydrogen.

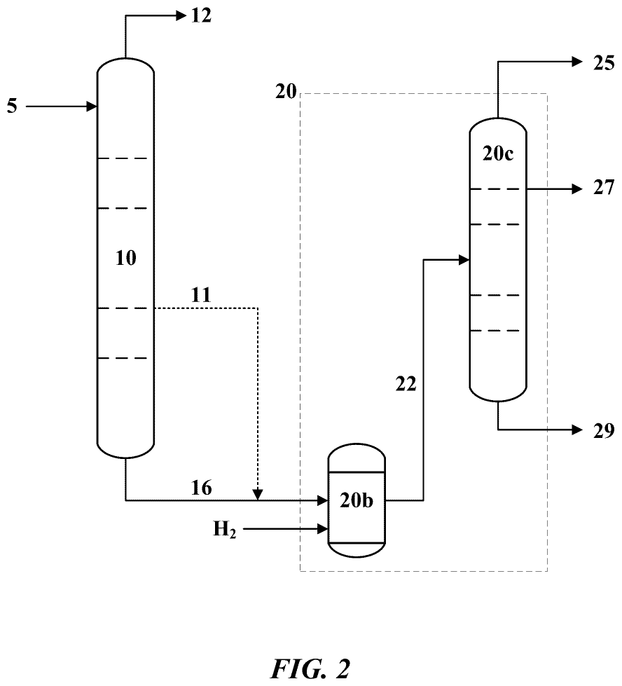

Turning to FIG. 2 in another embodiment of the present disclosure, the catalytic dechlorination zone 20 contains two vessels, the first being a reactor 20b, or reaction vessel, which contains one or more beds of dechlorination catalyst. Again, the catalyst promotes the conversion of organic chlorides to HCl and dechlorinated hydrocarbons (e.g., dechlorinated paraffins). The second of two vessels is a separation vessel 20c, such as a fractionation column, configured to separate an effluent stream 22 from the reactor 20b into a gaseous HCl-rich overheads stream 25, an n-butane-rich side-draw product stream 27 and a liquid alkylate-rich bottoms stream 29. In some aspects, the separation vessel is a conventional distillation column or a dividing-wall distillation column.

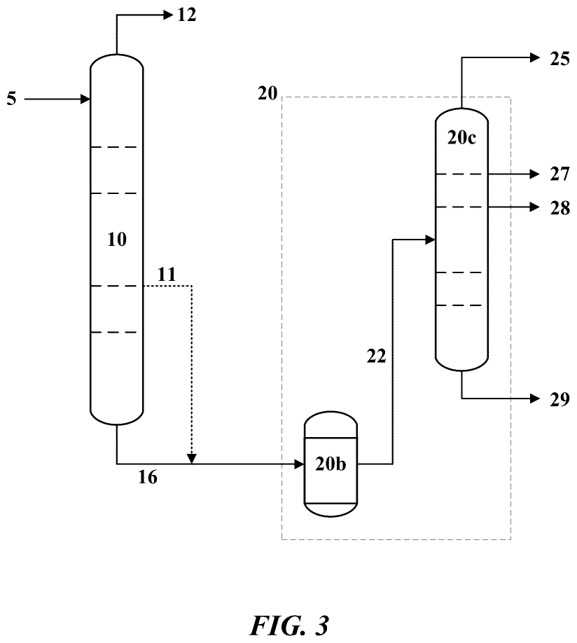

Turning to FIG. 3 in another embodiment of the present disclosure, the catalytic dechlorination zone 20 contains two vessels, the first being a reactor 20b, or reaction vessel, which contains one or more beds of dechlorination catalyst. Again, the catalyst promotes the conversion of organic chlorides to HCl and dechlorinated hydrocarbons (e.g., dechlorinated paraffins). The second of two vessels is a separation vessel 20c, such as a fractionation column, configured to separate an effluent stream 22 from the reactor 20b into a gaseous HCl-rich overheads stream 25, an n-butane-rich side-draw product stream 27, an isopentane-rich side-draw product stream 28 and a liquid alkylate-rich bottoms stream 29. The separation vessel may be a conventional distillation column or a dividing-wall distillation column.

In some aspects, the liquid n-butane-rich side draw product stream 27 containing HCl is recovered and passed to a n-butane isomerization unit as a feed to convert the n-butane to isobutane. The liquid isopentane-rich side draw product stream 28 containing HCl is recovered and recycled back to an alkylation reactor as a feed, where the isopentane is reacted with olefins to make additional alkylate. By taking a separate isopentane stream, the vapor pressure of the alkylate is controlled to a low Reid vapor pressure (RVP) level as desired. In some aspects, the alkylate-rich stream may be characterized as having a Reid vapor pressure of less than 6.5 psi, less than 5 psi, or less than 4 psi. RVP can be determined according to ASTM D323. The low RVP alkylate product may be used for blending into gasoline pools.

In some aspects, the liquid n-butane-rich side draw product stream and alkylate-rich product streams obtained by the present processes can have a chloride concentration of less than 200 wppm, or less than 100 wppm, or less than 50 wppm, as measured by ASTM D4929. Optionally, the n-butane-rich side-draw product stream or the alkylate-rich product stream may be passed to a small treater containing caustic solvent or adsorbent to further reduce the chloride concentration in these streams. Treated streams can have a chloride concentration of less than 10 wppm or even less than 5 wppm.

It should be appreciated and understood by those of ordinary skill in the art that various other components (e.g., valves, pumps, filters, coolers, etc.) were not shown in the drawings as it is believed that the specifics of same are well within the knowledge of those of ordinary skill in the art and a description of same is not necessary for practicing or understanding the embodiments of the present invention.

Any of the above lines, conduits, units, devices, vessels, surrounding environments, zones or similar may be equipped with one or more monitoring components including sensors, measurement devices, data capture devices or data transmission devices. Signals, process or status measurements, and data from monitoring components may be used to monitor conditions in, around, and on process equipment. Signals, measurements, and/or data generated or recorded by monitoring components may be collected, processed, and/or transmitted through one or more networks or connections that may be private or public, general or specific, direct or indirect, wired or wireless, encrypted or not encrypted, and/or combination(s) thereof; the specification is not intended to be limiting in this respect.

Signals, measurements, and/or data generated or recorded by monitoring components may be transmitted to one or more computing devices or systems. Computing devices or systems may include at least one processor and memory storing computer-readable instructions that, when executed by at least one processor, cause the one or more computing devices to perform a process that may include one or more steps. For example, the one or more computing devices may be configured to receive, from one or more monitoring component, data related to at least one piece of equipment associated with the process. The one or more computing devices or systems may be configured to analyze the data. Based on analyzing the data, the one or more computing devices or systems may be configured to determine one or more recommended adjustments to one or more parameters of one or more processes described herein. The one or more computing devices or systems may be configured to transmit encrypted or unencrypted data that includes the one or more recommended adjustments to the one or more parameters of the one or more processes described herein.

1. A process for processing a product stream comprising organic chlorides, n-butane and alkylate, the process comprising:
separating, in a separation zone, a feed stream comprising light hydrocarbons, propane, isobutane, organic chlorides, n-butane, and alkylate with C5+ hydrocarbons into overheads stream comprising light hydrocarbons, propane, and isobutane and a bottoms stream comprising organic chlorides, n-butane and alkylate; and
passing the bottoms stream to a catalytic dechlorination zone configured to convert the organic chlorides to HCl and paraffins under catalytic dechlorination conditions, the catalytic dechlorination zone comprising a vessel and being configured to provide a HCl-rich stream comprising the HCl and the paraffinsan n-butane-rich stream, and an alkylate-rich stream;
removing the HCl-rich stream from the catalytic dechlorination zone as a gaseous overheads product;
recovering the n-butane-rich stream from the catalytic dechlorination zone as a side-draw product; and
recovering the alkylate-rich stream from the catalytic dechlorination zone as a liquid bottoms product.

2. The process of claim 1, wherein the feed stream is an effluent from an ionic liquid catalyzed isoparaffin-olefin alkylation reaction zone.

3. The process of claim 1, wherein the first separation zone comprises a fractionation column.

4. The process of claim 3, wherein the fractionation column is a deisobutanizer column.

5. The process of claim 1, wherein the vessel in the catalytic dechlorination zone comprises a reactive distillation column.

6. The process of claim 5, wherein the reactive distillation column is a dividing-wall reactive distillation column.

7. The process of claim 5, wherein a dechlorination catalyst is disposed within the reactive distillation column.

8. The process of claim 1, wherein the catalytic dechlorination zone comprises at least two vessels, the first vessel comprising a reactor and the second vessel comprising a separation vessel.

9. The process of claim 8, wherein the separation vessel comprises a diving-wall column.

10. The process of claim 8, wherein a dechlorination catalyst is disposed within the reactor.

11. The process as in either claim 7 or 10, wherein the dechlorination catalyst is selected from the group consisting of silica, alumina, silica-alumina, magnesium oxide, titanium oxide, zinc oxide, zirconium oxide, activated carbon, a zeolite, and any combinations thereof.

12. The process of claim 11, wherein the dechlorination catalyst further comprises a precious metal selected from the group consisting of Pt, Pd, Ru, Re and any combinations thereof.

13. The process as in either claim 7 or 10, wherein the dechlorination catalyst is an ionic liquid comprising an organic cation and a halometallate anion and wherein the halometallate anion comprises a metal selected from Al, Ga, In, Mn, Fe, Co, Ni, Cu, Zn, or a combination thereof, and a halide selected from F, Cl, Br, I, or a combination thereof.

14. The process of claim 1, comprising recovering a side-draw stream from the separation zone, the side-draw stream comprising at least a portion of the n-butane, and passing the side-draw stream to the catalytic dechlorination zone.

15. The process of claim 1, comprising:
recovering a side-draw stream from the separation zone, the side-draw stream comprising at least a portion of the n-butane,
combining the side-draw stream with the bottoms stream to form a combined stream, and
passing the combined stream to the catalytic dechlorination zone.

16. The process of claim 1, wherein the organic chlorides comprise one or more of:
perchloroethylene, carbon tetrachloride, methyl chloride, methylene chloride, ethyl chloride, dichloroethane, dichloroethylene, trichloroethylene, dichloropropane, dichloropropene, sec-butyl chloride, tert-butyl chloride, and C5+ chlorides.

17. The process of claim 1, wherein the feed stream has a chloride concentration of 200 to 5000 wppm.

18. The process of claim 1, wherein the recovered alkylate-rich stream has a chloride concentration of less than 200 wppm.

19. The process of claim 15, further comprising passing the recovered alkylate-rich stream to a treater comprising caustic solvent or adsorbent to provide a treated alkylate-rich stream having a chloride concentration of less than 10 wppm.

20. The process of claim 1, wherein the recovered n-butane-rich stream has a chloride concentration of less than 200 wppm.

21. The process of claim 20, further comprising passing the recovered n-butane rich stream to a treater comprising caustic solvent or adsorbent to provide a treated n-butane rich stream having a chloride concentration of less than 10 wppm.

22. The process of claim 1, wherein catalytic dechlorination conditions include a temperature of from 150° F. to 650° F. (65° C. to 343° C.) and a pressure of from 10 to 1000 psig (69 kPa to 6.9 MPa gauge), in the presence or absence of hydrogen.

23. A process for processing a product stream comprising organic chlorides, n-butane and alkylate, the process comprising:
separating, in a separation zone, a feed stream comprising light hydrocarbons, propane, isobutane, organic chlorides, n-butane, and alkylate with C5+ hydrocarbons into an overheads stream comprising light hydrocarbons, propane, and isobutane and a bottoms stream comprising organic chlorides, n-butane and alkylate; and
passing the bottoms stream to a catalytic dechlorination zone configured to convert the organic chlorides to paraffins and HCl under catalytic dechlorination conditions, the catalytic dechlorination zone comprising a vessel and being configured to provide a HCl-rich stream, an n-butane-rich stream, an isopentane-rich stream, and an alkylate-rich stream;
removing the HCl-rich stream from the catalytic dechlorination zone as a gaseous overheads product;
recovering the n-butane-rich stream from the catalytic dechlorination zone as a first side-draw product;
recovering the isopentane-rich stream from the catalytic dechlorination zone as a second side-draw product;
recovering the alkylate-rich stream from the catalytic dechlorination zone as a liquid bottoms product.

24. The process of claims 23, wherein the recovered n-butane-rich stream contains hydrogen chloride and is passed to a n-butane isomerization unit as a feed.

25. The process of claim 23, wherein the recovered isopentane-rich stream contains hydrogen chloride and is recycled to the alkylation reactor as a feed.

26. The process of claim 23, wherein the recovered alkylate-rich stream has a Reid vapor pressure of less than 6.5 psi.

27. The process of claim 23, comprising recovering a side-draw stream from the separation zone, the side-draw stream comprising at least a portion of the n-butane, and passing the side-draw stream to the catalytic dechlorination zone.

28. The process of claim 23, comprising:
recovering a side-draw stream from the separation zone, the side-draw stream comprising at least a portion of the n-butane,
combining the side-draw stream with the bottoms stream to form a combined stream, and
passing the combined stream to the catalytic dechlorination zone.
